(19) United States
(12) Patent Application Publication
(10) Pub. No.: US 2018/0360459 A1
(43) Pub. Date: Dec. 20, 2018

(54) SURGICAL STAPLER

(71) Applicants: J.M.C. S.R.L., Pallermo (PA) (IT); TSALYS LIMITED, Cheung Sha Wan-Kowloon (HK)

(72) Inventors: Federico BILOTTI, Aprilia (LT) (IT); Giovanni CIOTTA, Palermo (PA) (IT)

(73) Assignee: Giovani CIOTTA, Palermo (PA) (IT)

(21) Appl. No.: 15/739,455

(22) PCT Filed: Jul. 1, 2016
(86) PCT No.: PCT/IB2016/053973

§ 371 Date: Dec. 22, 2017

(30) Foreign Application Priority Data
Jul. 2, 2015 (IT) 102015000029890

(51) Int. Cl. A61B 17/115; A61B 17/11; A61M 29/02

(52) U.S. Cl. CPC A61B 17/1155; A61B 2017/00389; A61M 29/02; A61B 17/1114

(57) ABSTRACT

A half-circular double stapler which allows application of wire stitches and resection of the rectal prolapse without the need of using two circular staplers is provided. A fastener-pushing system is divided into two bodies which can be activated independently, each body having a semi-circular shape. The stapler has a gun-shaped handgrip, a pair of triggers and a cursor arranged laterally for a quick closure of anvil and feeder. The stapler also has a switching button arranged between the first and the second fastener-pushing bodies.

### TECHNICAL FIELD OF THE INVENTION

The present invention relates to a surgical stapler, in particular of circular type.

The stapler is specifically suitable for the surgical treatment of front and/or rear rectal prolapse of haemorrhoidal prolapse, by applying staples.

### BACKGROUND

Nowadays haemorrhoids represent an important sanitary problem and the frequency of surgical operations for their reduction has been growing quickly. The most used intervention methods are based upon the use of circular surgical staplers.

As it is known, the surgical stapler is an instrument for applying suturing elements between two tissue excerpts or fragments, in particular for inserting so-called “fasteners” or stitches.

The surgical staplers known in the art include, distally, a feeder suitable to expel one or more staples and a corresponding “anvil” or contrast. During use, the surgeon arranges the two tissue portions to be sutured between the feeder and the anvil and actuates the instrument so as to determine the expulsion of the fasteners. The fasteners penetrate the two tissue portions to be joined and abut the anvil.

In the treatment of haemorrhoids, the prolapsed tissue is kept between the anvil and the feeder of a circular stapler and a knife performs the removal of the exceeding tissue while the remaining portions are sutured.

In case—which is the most frequent one—of both front and rear rectal prolapse, the current surgical method provides a removal/suturing in two steps. In particular, two circular staplers are used which perform separately, in sequence, first of all the resection of the rectum front prolapse and then of the rear one.

An example of the just described technique is the so-called “STARR” (Stapled Trans-Anal Rectal Resection) procedure.

The use requested for the known procedures described above, both in terms of cost and performing time, tends to be high, and this is indeed due to the two-step performing and the use of two staplers.

EP2130498 describes a linear stapler comprising two sets of cartridges which can be actuated in sequence, each one by means of a respective lever. However, such stapler does not allow a selection of the set of cartridges to be actuated by the operator, nor it allows to obtain, selectively and separately, a position for approaching a cartridge to the anvil and a subsequent configuration for expelling the staples. Furthermore, such stapler does not allow to perform a resection and a circular anastomosis between two portions of tubular intestine even in an independent way.

### SUMMARY OF THE INVENTION

The technical problem placed and solved by the present invention is then to provide a circular surgical stapler allowing to obviate the drawbacks mentioned above with reference to the known art.

Such problem is solved by a stapler according to claim 1.

Advantageously, the invention also provides a Circular Anal Dilator (or Circular Anoscope Device, CAD) suitable for the use in association with said stapler, and in particular specifically configured to be used together with the stapler.

Preferred features of the present invention are set forth in the depending claims.

The invention provides a circular stapler equipped with a first fastener-pushing device and a second fastener-pushing device which can be actuated independently from one another for expelling stitches, or staples, at respective selected portions of prolapsed tissue, in particular a front portion and a rear portion of the rectum.

Advantageously, each fastener-pushing device has a half-circular shape, corresponding to half of the suturing line which can be performed with the stapler.

A respective half-circular blade, or knife, can be associated to each one of said fastener-pushing devices. Alternatively, one single circular blade, or knife, can be provided, which is actuated together with each one of the two fastener-pushing bodies.

In other words, the invention provides a half-circular double stapler, or double-activation stapler, allowing the application of metal stitches and the resection in a differentiated and independent way on two half-circumferences of prolapsed tissue.

The instrument of the invention eliminates the need for using two circular staplers in the procedures for treating haemorrhoids discussed by referring to the known art. In particular, it offers to divide the fastener-pushing system—and preferably even of the cutting system—from circumferential into half-circular.

Preferably, the stapler comprises means to determine the approaching of a (fastener-bearing) feeder to a distal anvil and means for causing a subsequent compression between such two elements of the tissue to be sutured. Advantageously, such means is suitable to perform a quick closure of the anvil on the cartridge, and preferably it comprises a cursor arranged aside the stapler, in particular on a handpiece thereof, and a proximal trigger dedicated to the closure of the anvil on the feeder (or viceversa).

In an advantageous embodiment, the half-circular stapler of the invention comprises a plurality of openings, preferably four, formed at the base of a distal head thereof, that is of the feeder, for externalizing traction wires applied to the prolapsed tissue before suturing.

According to a preferred alternative embodiment, the stapler comprises a handpiece, shaped like a gun. Advantageously, the surgeon can grasp the stapler with his/her hand's palm at such handpiece and activate one or more triggers for actuating the fastener-pushing devices, and possibly the cutting means, with the fingers.

Furthermore, the surgeon can easily rotate the stapler to perform a selective intervention on front, rear or side portions of the prolapse.

Advantageously, the stapler can be used by right-handed or left-handed operators in the same way.

For such gun-shaped handgrip, since it is associated to staplers even of different type with respect to the herein considered one and subject of the following claims, a separate protection could be requested.

The stapler of the invention can allow to maximize the visibility and/or the control during the operation, in particular by allowing to approach the feeder to the anvil wherein the latter is kept still. Such motion is opposite to that of the circular staplers of known art.

As mentioned above, advantageously a multi-functional anal dilator (CAD) is associated to the stapler, that is an anal dilator having a configuration which can be modified depending upon the—front, rear or lateral—suture line to be performed.

Advantageously, such dilator allows to expose the front prolapse of the patient independently of the rear and lateral one.

The stapler according to the invention—in particular when assisted by the above-mentioned dilator—allows a continuity of the surgical procedure, that is it does not request to extract the instrument between the steps of suturing/resecting the rear prolapse. In other words, it allows to perform the whole surgical procedure with one single instrument, without the need of extracting from the rectum for treating separate—front and rear—portions of prolapse.

Therefore, the proposed stapler allows to reduce cost and time of the operation. Moreover, it results to be extremely versatile with respect to specific intervention needs.

Still, the anastomotic ring resulting after suturing is more flexible, as fewer staples can be applied to the tissue.

In addition, the stapler can allow to reduce the clinical complications of the operation, in particular in terms of blooding and residual haemorrhoidal prolapse.

Other advantages, features and use modes of the present invention will result evident from the following detailed description of some embodiments, shown by way of example and not for limitative purpose

### BRIEF DESCRIPTION OF THE FIGURES

The figures of the enclosed drawings will be referred to, wherein:

FIG. 1A shows a side view of a preferred embodiment of the surgical stapler according to the present invention, suitable for an operation for treating rectal haemorrhoids;

FIG. 1B shows the same view of FIG. 1B, wherein some of the inner components are made visible;

FIG. 2A shows a perspective view of a distal portion of the stapler of FIG. 1A, wherein an outer casing has been removed so as to show some inner components and wherein an anvil and related fastener-pushing devices are spaced apart;

FIG. 2B shows a side view of the distal portion of FIG. 2A, wherein an outer casing has been removed so as to show some inner components and wherein said anvil and fastener-pushing devices are approached;

FIGS. 3A and 3B relate to means for actuating the fastener-pushing devices, by showing them at a handpiece of the stapler in a first configuration, in a rear perspective view of outer components and inner components, respectively;

FIGS. 3C and 3D relate to means for actuating the fastener-pushing devices, by showing them at a handpiece of the stapler in a second configuration, in a rear perspective view of outer components and inner components, respectively;

FIGS. 4A to 4F relate to an anal dilator apt to be used in association with the stapler of FIG. 1A, by showing respectively: a front perspective view of the dilator in assembled configuration; a perspective view, a plan view and a side view of a base thereof; a front perspective view of a flap thereof; and a front perspective view of a shutter which can be used together with the dilator;

FIG. 5 shows a side view of the stapler of FIG. 1A, wherein some of the inner components are made visible, in a first configuration of preparation to the surgical operation;

FIGS. 6A and 6B relate to an initial phase of the operation, wherein a first set of traction wires is applied to a prolapsed tissue, such figures showing each one a front view respectively of the dilator of FIG. 4A and of portion of the feeder of the stapler of FIG. 1A;

FIGS. 7A and 7B relate to a subsequent initial phase, wherein a second set of traction wires is applied to the tissue, such figures showing each one a front view respectively of the dilator of FIG. 4A and of portion of the feeder of the stapler of FIG. 1A;

FIG. 8 relates to another intervention phase, wherein a variant of the dilator of FIG. 4A is arranged for the resection of a front prolapse, such figure showing a side view of such dilator;

FIG. 9 shows a side view of the stapler of FIG. 1A, wherein some of the inner components are made visible, in another configuration preparatory to the execution of the surgical operation wherein feeder and anvil have been approached;

FIG. 10 shows a side view of the stapler of FIG. 1A, wherein some of the inner components are made visible, in an operating configuration for executing the surgical operation wherein feeder and anvil are approached to each other;

FIG. 11 shows a side view of the stapler of FIG. 1A, wherein some of the inner components are made visible, in a subsequent configuration for executing the surgical operation wherein the staples are applied to a front portion of prolapsed tissue;

FIGS. 12 and 13 both show a side view of a variant of the dilator of FIG. 4A, in a respective, additional phase of the operation preparatory to the suturing of a rear portion prolapsed tissue;

FIG. 14 shows a top view of the stapler of FIG. 1A, in a configuration for actuating switching means between the actuation of a first and a second fastener-pushing device;

FIGS. 15A and 15B both show a perspective view of a portion of a surgical stapler according to an embodiment variant of the present invention, respectively in a configuration for locking and unlocking a relative anvil;

FIGS. 16A and 16B show some inner components of the stapler of FIGS. 15A and 15B, in the same views and configurations of these last two figures; and

FIG. 17 shows a front view of some inner components of the stapler of FIGS. 15A and 15B.

The sizes and curvatures represented in the above-illustrated figures are to be meant as pure example and they are not necessarily shown in proportion.

### DETAILED DESCRIPTION OF PREFERRED EMBODIMENTS

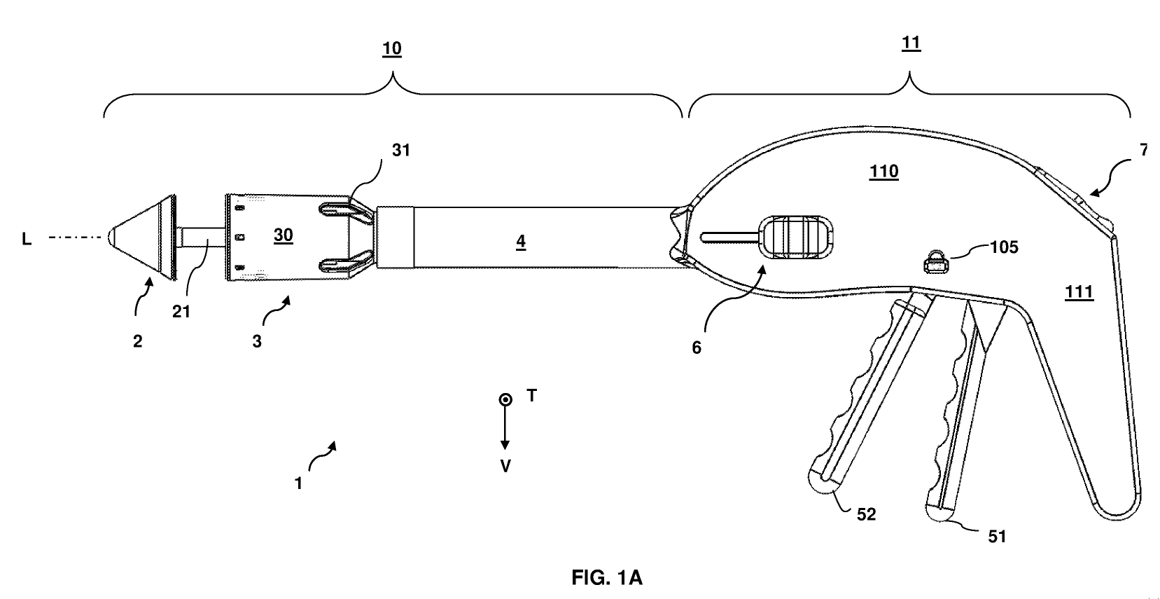

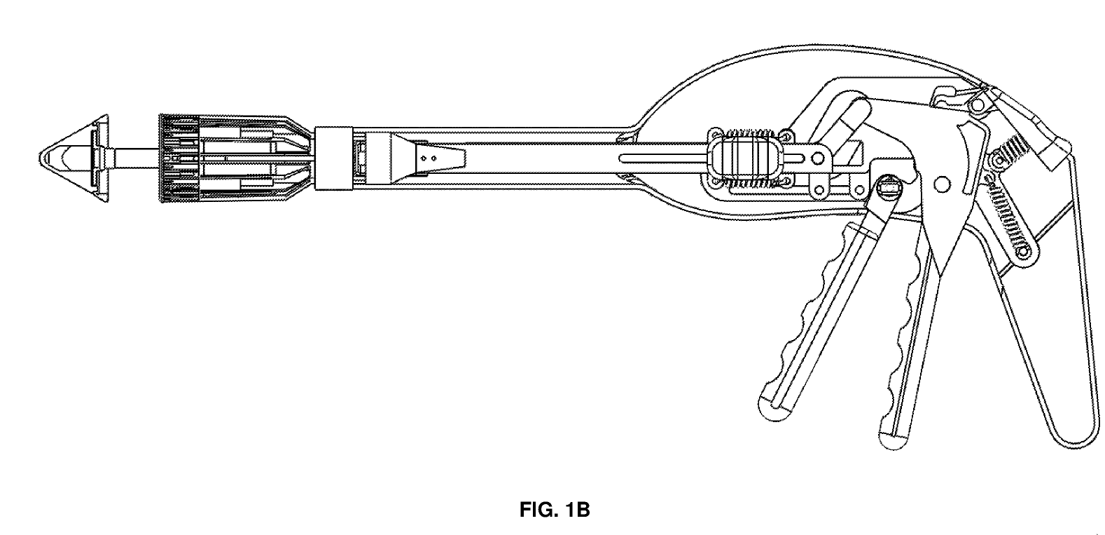

By firstly referring to FIGS. 1A and 1B, a surgical circular stapler according to a preferred embodiment of the invention is designated as a whole with 1.

The stapler 1 is devised to be used in operations of anastomosis or suturing of hollow tubular organs and more specifically for the treatment of the rectal prolapse or haemorrhoids.

The stapler 1 mainly comprises an elongated distal portion 10, suitable to be inserted in the rectum, and a proximal portion 11, which can be handled by the operator during the operation.

The stapler 1, and in particular the distal portion thereof 10, extends according to a prevailing longitudinal direction L, which corresponds even to the direction for inserting into the rectum, as well as, as it will be said shortly, to a direction for expelling staples or stitches. In order to improve understanding of the invention, in FIG. 1A even the two transversal directions orthogonal to the longitudinal direction L, which transversal directions are designated with T (orthogonal to the sheet) and V, have been shown.

The distal portion 10 is mainly formed—by following an order of arrangement from distal to proximal—by an anvil 2, a feeder, or cartridge, 3 and by an operating segment 4 for the connection to the proximal portion 11.

The proximal portion 11 consists of a handpiece 110 which can be grasped by an operator.

Each one of the sofar illustrated components will be now described in greater detail.

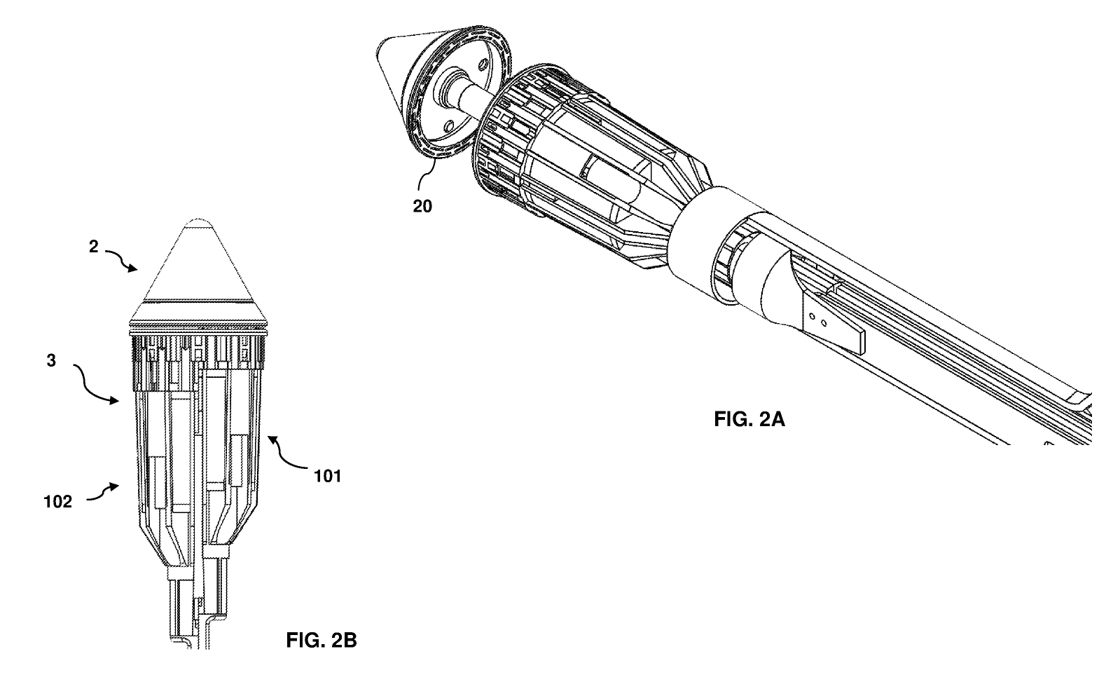

By also referring to FIGS. 2 and 2A, the feeder 3 is apt to the expulsion of fasteners for their penetration into the tissue. It mainly comprises a first and a second fastener-pushing device, designated respectively with 101 and 102, which can be actuated independently from one another for suturing a respective portion of prolapsed tissue. In the configuration of FIG. 2A, the first device 101 results to be in a distally advanced position, that is it results to be actuated, to perform the suturing of a front portion of rectal prolapse. The term “front” in the present context is referred to the body of the patient, which typically assumes a lithotomy position during the operation, wherein the front portion of the body, and then of the rectum, corresponds to a right portion of the herein illustrated drawings of the the stapler.

Each one of the devices 101 and 102 is suitable to determine the expulsion of a respective plurality of fasteners in the prolapsed tissue according to a half-circular profile for the application of the staples. Advantageously, each device 101, 102 allows the application of a double series of staples, arranged according to concentric half-circumferences.

The feeder 3 comprises an outer casing or skirt 30, at which a plurality of openings 31, preferably four, are obtained. As it will be illustrated hereinafter, each one of such openings 31 is configured to allow passage to the outside of one or more traction wires applied to the prolapsed tissue. Advantageously, every one of the openings 31 has an elongated shape in the longitudinal direction L and extend even at a portion of feeder 3 having a taper in the proximal direction.

The anvil 2 is configured to act as abutment for the fasteners expelled from the just-described fastener-pushing devices 101, 102 and it has, similarly to the staplers of known art, a plurality of seats 20 dedicated to house the legs of the staples to bend them.

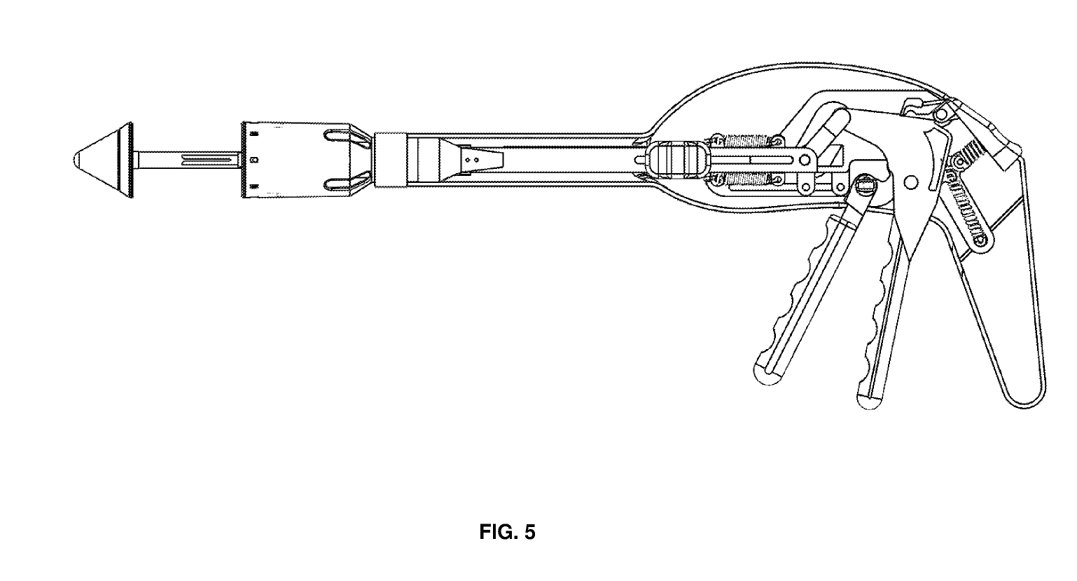

As it will be explained even hereinafter, feeder 3 and anvil 2 are mutually movable, that is they can be mutually approached/spaced apart, according the direction L. In particular, such components 2 and 3 can translate to assume a plurality of mutual positions, and in particular:
a first maximally spaced-apart rest configuration, shown in FIG. 5;
a second spaced-apart configuration, preparatory to the suturing, shown in FIGS. 1A, 1B, 2A and 9, wherein the anvil 2 has been retracted, that is translated in the proximal direction, to approach the feeder 3 and wherein a prolapsed tissue can be inserted between such components; and
a third operating configuration for suturing, shown in FIGS. 2B, 10 and 11, wherein the feeder 3 is made to abut the anvil 2 and is suitable to expel the staples.

By still referring to FIGS. 1A to 2B, the passage from the above-illustrated first configuration to the second configuration is obtained by means of dedicated actuation means, determining an approaching of the anvil 2 to the feeder 3. In particular, the stapler 1 includes an anvil-carrying shaft 21 extending longitudinally within the distal portion 10 and it is proximally associated to said actuation means, the latter arranged at the handpiece 110.

In the present embodiment, the actuation means comprises at least a cursor 6 mounted on the anvil-carrying shaft 21 and which can be actuated by the operator to determine a sliding thereof in direction L. Therefore, a shifting of the cursor 6 in the proximal direction, that is a retraction, determines a corresponding retraction of the anvil-carrying shaft 21 and then of the anvil 2 integral to the latter. Advantageously, in the illustrated example two cursors of the above-mentioned type are provided, arranged bilaterally on the handpiece 110. This allows the operator to choose the hand with which he/she has to act.

The passage from the above-mentioned second configuration to the third configuration is determined by other dedicated actuation means which in this case determine an approaching of the feeder 3 to the anvil 2, that is a translation of the first in the distal direction. Such shifting is obtained by means of the actuation of a first trigger 51, arranged at the proximal portion 11 and operatively associated to the handpiece 110.

The expulsion of the fasteners from one of the two fastener-pushing devices 101 and 102 of the feeder 3 is determined by additional dedicated actuation means. In particular, a second trigger is provided 52, arranged at the proximal portion 11 too, distally with respect to the first trigger 51, and operatively associated to the handpiece 110.

The triggers 51 and 52 are both equipped with an ergonomic handgrip, with four recesses each one suitable to be engaged by a finger of the operator.

The triggers 51 and 52 can be both actuated in rotation, according to a rotation axis extending in transversal rotation T, that is orthogonal to the longitudinal direction L. Each trigger 51, 52 is operatively connected to additional mechanical components, in particular levers, apt to act on respective distal components to actuate them.

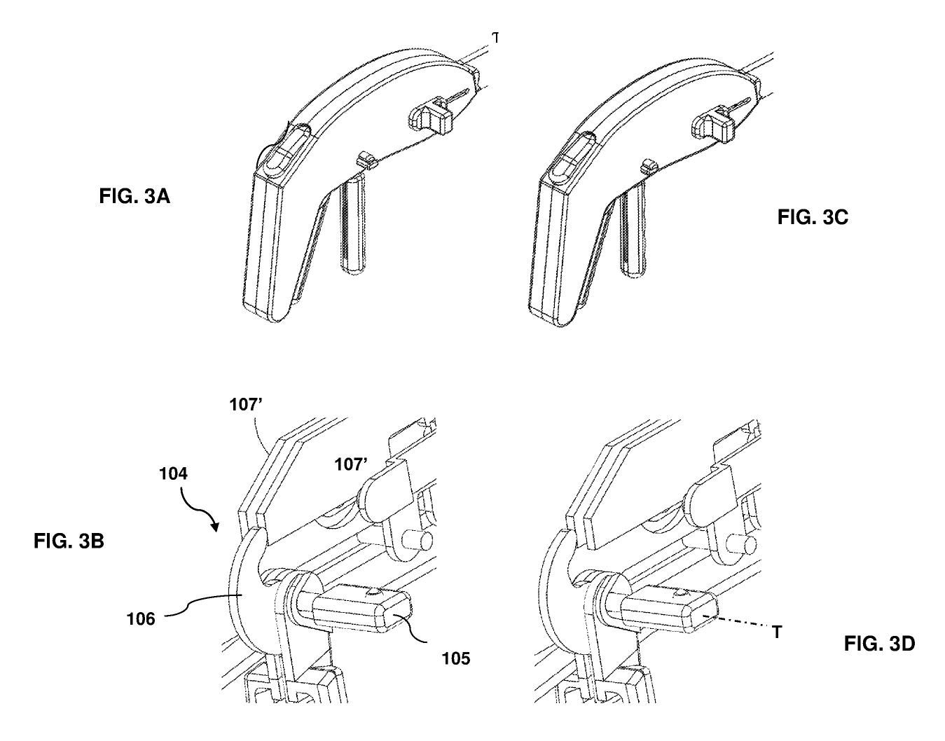

By referring to FIGS. 3A to 3D, the stapler 1 further comprises switching means 104, configured to allow a selection between the two fastener-pushing devices 101 and 102, that is to determine which one of the two is actuated, and then it expels the staples in the tissue, when the operator acts on the second trigger 52.

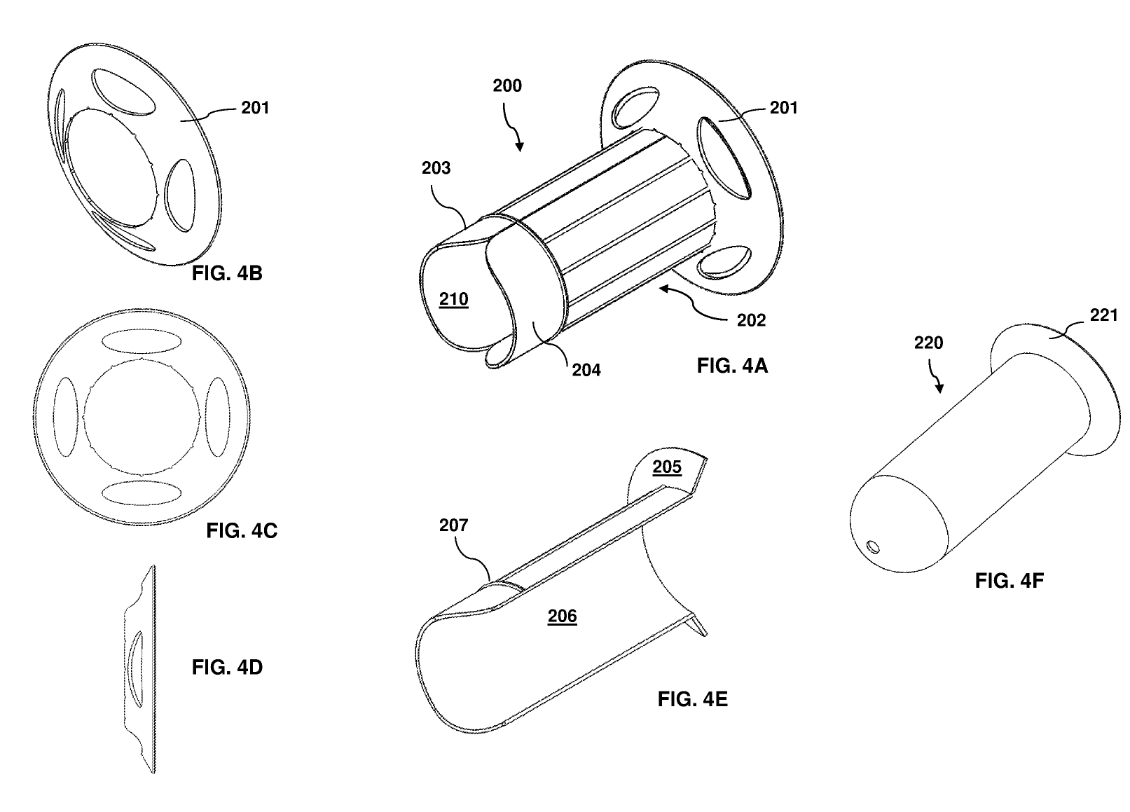

The switching means 104 comprises a proximal switch 105 which can be actuated by the operator and arranged at the handpiece 110. In the present example, the switch 105 can be actuated in translation according to the transversal direction T orthogonal to the longitudinal direction L. The switch 105 is integral to a pushing element 106, substantially shaped like a hook in the considered example. The latter, in turn, is suitable to exert a longitudinal push, in particular a pulse for expelling the staples, one of two actuating longitudinal rods 107 and 107′. Such rods are distally both associated to a respective fastener-pushing device 101 or 102. Then, depending upon the most projecting (FIG. 3A, 3B) or innermost (FIG. 4A e 4B) position of the cursor 105, the pushing element 106 acts on one of the above-mentioned two rods 107 and 107′.

By referring again to FIGS. 1A and 1B, the stapler 1 further comprises resetting means, configured to bring the triggers 51 and 52 back to a rest configuration wherein they are ready to be actuated by an operator as described above. In the present example, the resetting means comprises a lever, or button, 7 arranged on the rear side on the handpiece 110 and operatively associated to additional mechanical components suitable to bring back the triggers 51 and 52 in a distally advanced position.

Advantageously, in the present example the proximal handpiece 110 comprises a portion of handgrip 111 substantially shaped like a gun and which can be grasped by the operator with the palm of the hand.

The stapler 1 is specifically suitable to the use with a circular anal dilator 200, thereto the FIGS. 4A to 4F refer.

The dilator 200 mainly comprises:
an enlarged base 201 apt to abut the anus;
a hollow and elongated main body 202 apt to be inserted into the rectum, which main body 202 has substantially cylindrical geometry and it extends according to a longitudinal axis which, upon use, corresponds to the direction L.

The main body 202 has a lateral skirt defining an internal longitudinal cavity 210. Such lateral skirt is formed at least partially by a plurality of longitudinal sliding sectors, in particular two sectors 203 and 204 in the present example.

Each longitudinal sector 203 and 204 is slidingly associated to the base 201, so as to result to be selectively retracted during a surgical procedure to allow the insertion of tissues within said longitudinal cavity 210.

In the present example, each sector 203, 204 consists of a longitudinal element 206 defining a portion of the lateral skirt of the dilator 200 and by an abutment element, or flap, 205, apt to abut on a face of the base 201. Such abutment coupling defines a stop in the distal forward motion of the sector 203, 204 within the rectum of the patient.

Advantageously, in the present embodiment each sector 203, 204 has a peripheral rib 207, or equivalent locking means, to stop the sliding of the sectors outgoing from the base—that is to act as stop—and keep an integral structure of the dilator 200.

The dilator 200 is suitable to be used in combination with a shutter 220, shown in FIG. 4F. The latter can be removably inserted in the cavity 210 of the dilator 200. Even the shutter 220 has a base flap 221 apt to abut on the base 201 of the dilator 200 to determine a stop.

A surgical operation for removing prolapsed rectal tissue, in particular haemorrhoids, will be now illustrated, performed with the above-described stapler 1.

First of all, in the present example the patient assumes the so-called lithotomy, that is supine, position.

In a preliminary step of the operation, the dilator 200 is inserted in the anus together with its shutter 220, in a configuration wherein both longitudinal sectors 203 and 204 are in position maximally advanced in the distal direction and then the flaps 205 are abutted onto the base 201 (configuration of FIG. 4A).

In a step subsequent to the insertion, the shutter 220 is removed and the dilator 200 is fastened to the anus skin by way of suture wires or the like. The locking position of the dilator 200 has to be such that the longitudinal sectors 203 and 204 are one in front position (that is at twelve o'clock) and the other one in rear position (that is at six o'clock).

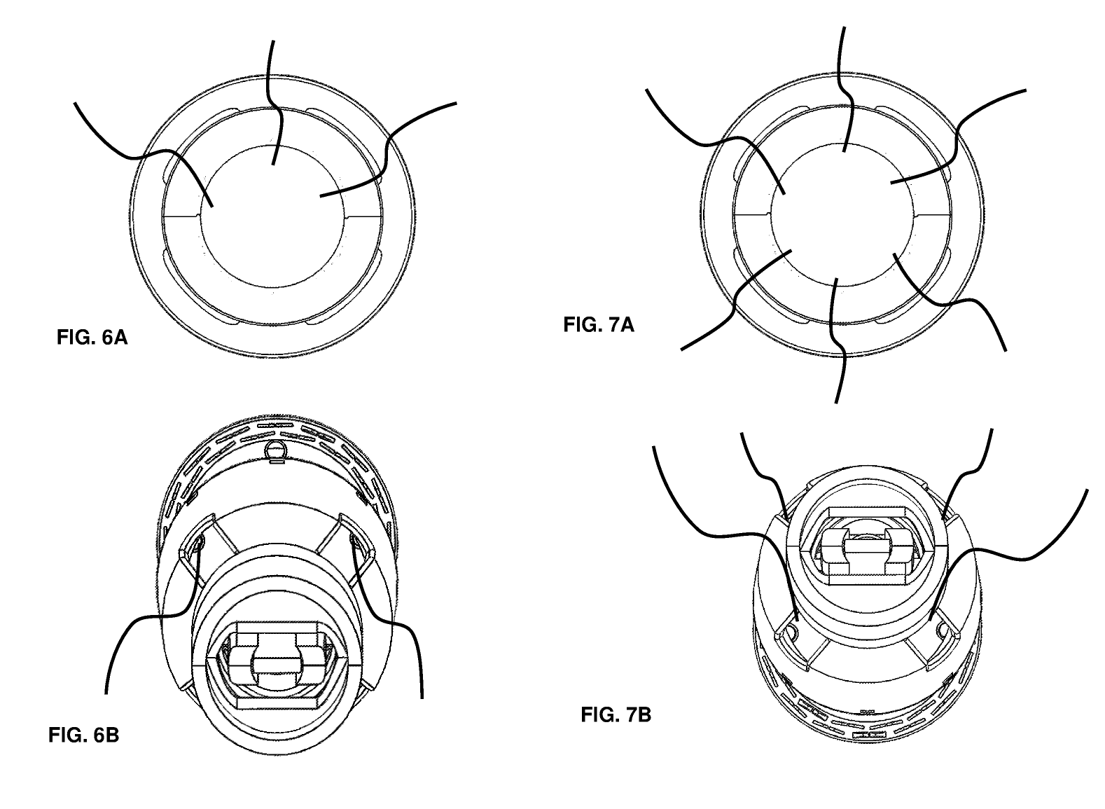

At this point, as shown in FIG. 6A, three traction wires are applied to the front rectal prolapse, in particular at ten, twelve and two o'clock, similarly to the configuration of a half-parachute.

Subsequently, as shown in FIG. 7A, three additional traction wires are applied to the rear rectal prolapse, in particular at eight, six and four o'clock, by completing the parachute.

At this point the stapler 1 can be inserted in the dilator 200, in the first rest configuration shown in FIG. 5, that is with its own maximally opened distal head defined by the anvil 2 and by the feeder 3. In such configuration, the cursor(s) 6 is(are) in the position maximally advanced in the distal direction.

As shown in FIGS. 6B e 7B, the front traction wires are made to pass through the two openings 31 on the right of the feeder 3 and the rear wires through the two openings 31 on the left.

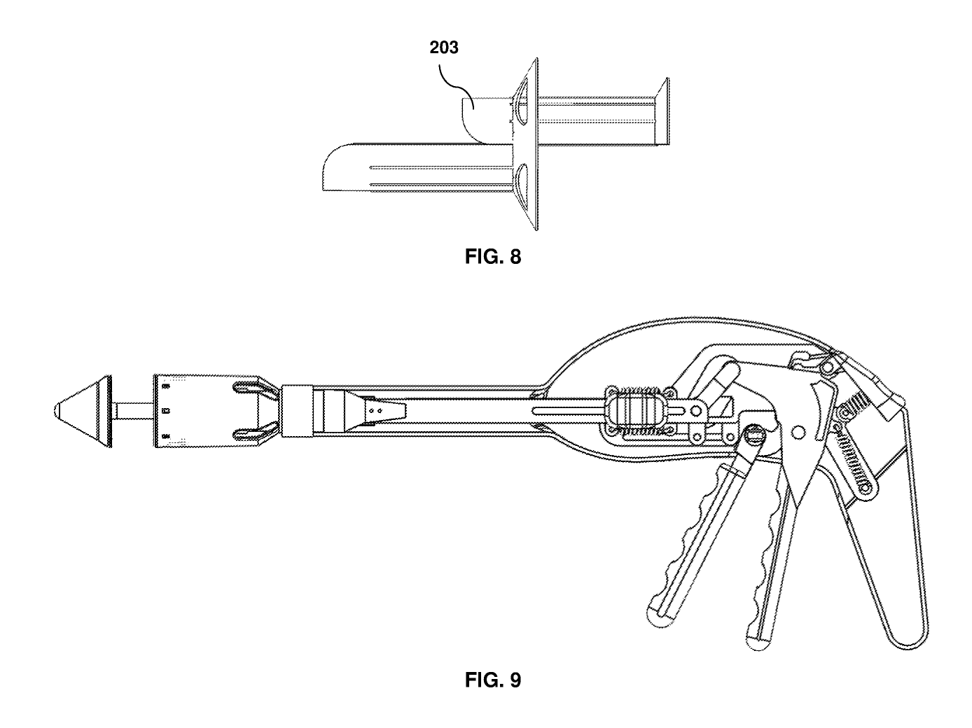

As shown in FIG. 8, at this point the front longitudinal sector 203 (at twelve o'clock) is extracted from the dilator 200 in order to make the front prolapse fall inside the cavity 210 and between anvil 2 and feeder 3.

The rear sector 204 is left inserted to protect the rear prolapse.

At this point the front wires are drawn, by guaranteeing that the prolapse is dragged into the compartment of the stapler 1 defined between anvil 2 and feeder 3.

Without drawing the rear wires, the anvil 2 is retracted proximally by acting on the cursor(s) 6. The stapler 1 then is in the second configuration preparatory to suturing, as shown in FIG. 9. The distance between anvil and feeder, in such configuration, can be about 2 cm (between the two faced surfaces of such components).

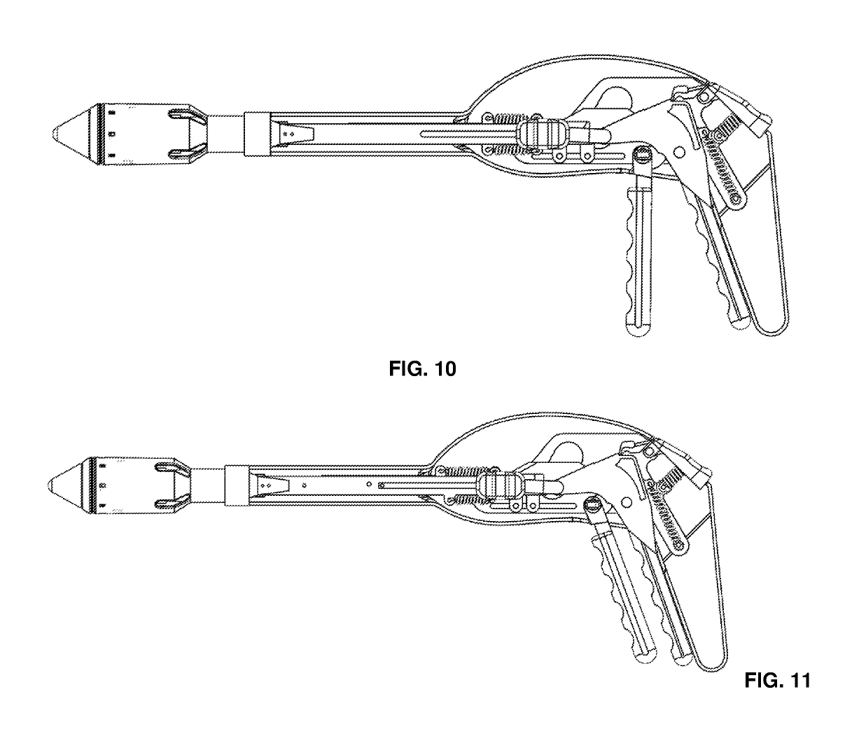

In a subsequent step exemplified in FIG. 10, the operator acts on the first trigger 51 so as to bring the stapler 1 in the third configuration mentioned above. In this way, the prolapsed front tissue is compressed between anvil 2 and feeder 3.

According to a well-known operation mode, after 15 seconds the operator acts onto the second trigger 52, thus causing cutting of the prolapse by means of a (not shown) blade, and expulsion of the fasteners from the first fastener-pushing device 101. A cut and a suturing are thus carried out along an operation profile extending for a circumference of about 180°. This step is exemplified in FIG. 11.

In a subsequent step, the triggers 51 and 52 are brought back to the not rotated initial position by acting onto the button 7.

Then, the cursor(s) 6 are operated so as to bring the stapler 1 back to the rest configuration, wherein the anvil 2 is maximally advanced in the distal direction.

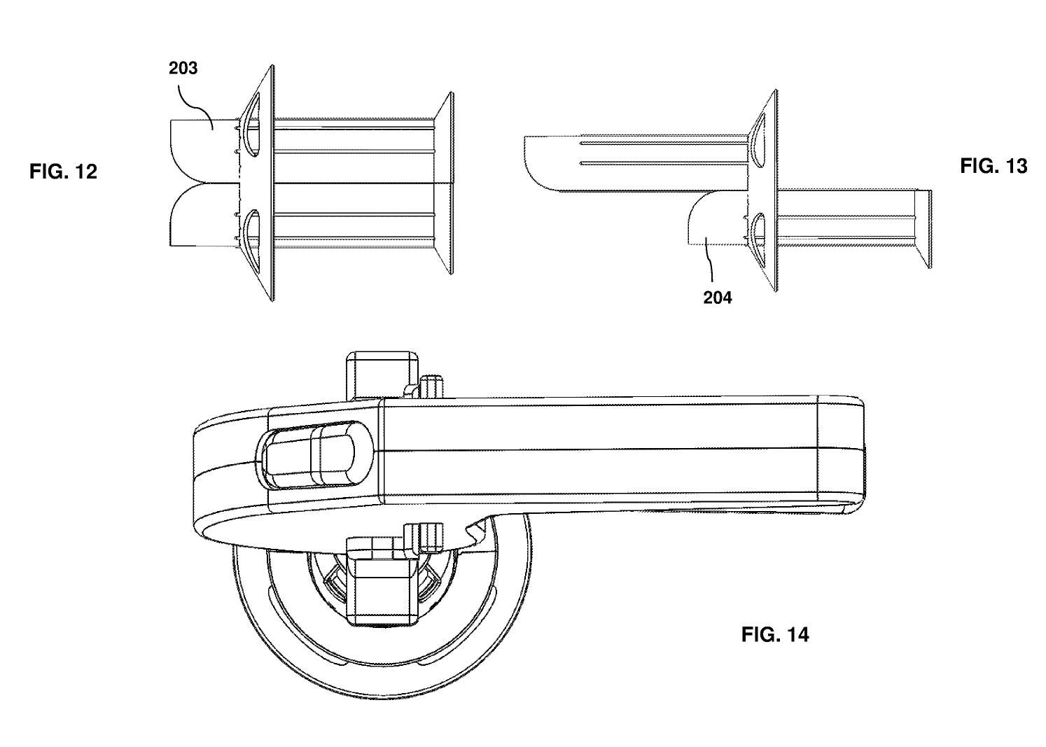

As shown in FIG. 12, the rear longitudinal sector 204 (at six o'clock) is retracted to make the rear prolapse to fall into the compartment defined between anvil 2 and feeder 3.

Furthermore, as shown in FIG. 13, the front sector 203 is inserted inside the anus to protect the already performed front suturing line.

At this point, the rear wires are drawn, thus making the rear prolapsed tissue to slide in the above-mentioned compartment of the stapler 1, similarly to what is previously done for the front prolapse.

The procedure is then carried out analogously to what already illustrated by referring to the front prolapse. As exemplified in FIG. 14, at the latest after activating the first trigger 51, the operator actuates the switch 105 so as to determine the subsequent actuation of the second fastener-pushing device 102.

The stapler 1 is subsequently extracted from the patient anus and, if necessary, haemostasis stitches are applied.

The dilator 200 can be extracted by proceeding to retract firstly the longitudinal sectors 203 and 204 and then the whole instrument.

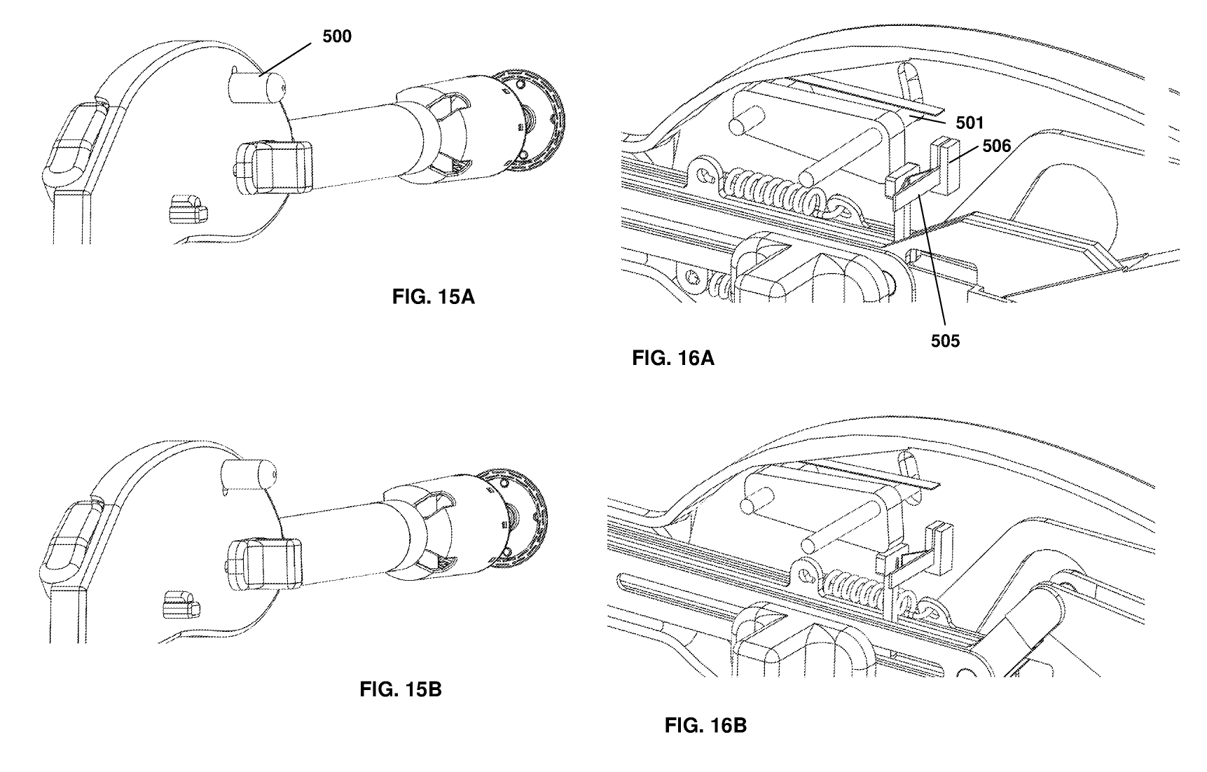

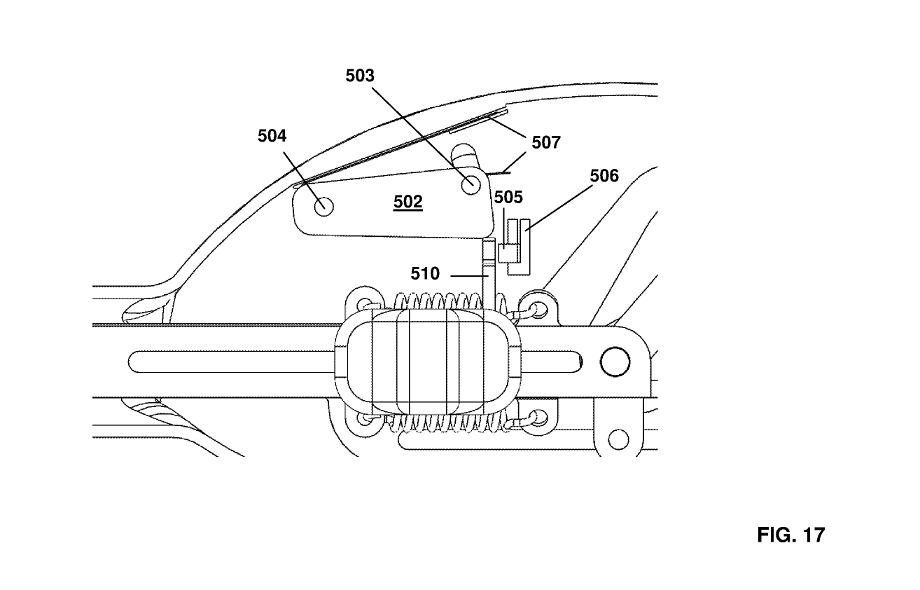

FIGS. 15A to 17 relate to a variant of the stapler of the invention, wherein the stapler comprises a system for selectively locking the anvil in the above-mentioned intermediate (retracted) configuration.

Such system comprises a button, or lever 500—or other actuating element—which can be actuated by the operator to determine the locking of the anvil in retracted position or the unlocking thereof. In particular, FIGS. 15A and 16A relate to a locked configuration and FIGS. 15B and 16B to an unlocked configuration.

The system further comprises an inclined guide 502, or oscillating body, which is hinged at its own end portions respectively to a frame or casing of the stapler and to a pin or extension 501 of the button 500. The revolving connection means implementing such hinging is designated with 503 and 504. A “V”-shaped spring 507—or equivalent spring contrast means—acts on the inclined guide 502.

They system further comprises a pushing spring 505, or other spring contrast element, acting on the orthogonal extension 510 of the anvil-carrying shaft, which spring 505 is received in a support 506, in particular of fork-like type.

The pushing spring 505 exerts an elastic action which tends to bring the extension 510 in a (distally) advanced position of the anvil. In other words, the rest position of such spring corresponds to an advanced position of the anvil.

The “V”-shaped spring 507 exerts an elastic action on the tilted guide 502 in the sense of forcing it in a lowered position wherein it interferes with the extension 510, by locking the anvil in pulled-back position.

In the shown example, a shifting upwards of the button 500 determines an unlocking of the anvil.

Based upon an embodiment variant, a mechanism may be added so as to adjust the stitch height, similar to what is available for some known “cutting-and-sewing” linear staplers. This mechanism allows the operator to pre-select the desired height of the closed stitch.

The present invention has been sofar described with reference to preferred embodiments. It will be appreciated that other embodiments belonging to the same inventive idea may exist, as defined by the scope of protection of the claims set forth below.

1. A circular surgical stapler, suitable for use in operations of anastomosis or of suturing tubular hollow organs for application of suture fasteners to a tissue, comprising:
a distal feeder configured to allow the expulsion of fasteners for their penetration into the tissue,
which distal feeder in turn comprises a first and a second fastener-pushing device each one suitable to determine the expulsion of a respective plurality of fasteners in the tissue, which first and second fastener-pushing device can be actuated independently from one another to suture a respective portion of tissue;
an anvil arranged distally to said distal feeder and configured to act as an abutment for fasteners expelled from said first and second fastener-pushing device,
wherein said distal feeder and said anvil are configured to assume a spaced-apart preparing configuration, wherein a prolapsed tissue can be introduced therebetween, and an abutted suturing configuration, wherein fasteners can be expelled in the tissue;
a proximal handpiece which can be grasped by an operator;
a first proximal trigger, operatively connected to said proximal handpiece and configured to determine an abutment of said distal feeder on said anvil in said suturing configuration;
an actuator for actuating said first and second fastener-pushing device, which actuator comprises a second proximal trigger arranged at said proximal handpiece and operatively connected thereto, which second proximal trigger is configured to determine an expulsion of fasteners from one of said fastener-pushing devices; and
a switch, arranged at said proximal handpiece too and configured to allow a selection between said first and second fastener-pushing device so that said actuator actuates said first or said second fastener-pushing device, which switch can be actuated in translation.

2. The circular surgical stapler according to claim 1, wherein said fastener-pushing devices each have a half-circular shape corresponding to half of a suturing line which can be performed with the stapler.

3. The surgical stapler according to claim 1, wherein said switch can be actuated in translation according to a direction substantially orthogonal to a longitudinal direction of approaching/spacing apart of said distal feeder and anvil.

4. The circular surgical stapler according to claim 1, wherein said first proximal trigger is arranged proximally with respect to said second proximal trigger (52).

5. The circular surgical stapler according to claim 1, comprising resetting means configured to return said first and second proximal trigger in a rest configuration wherein they are ready to be actuated by an operator, which resetting means is arranged at said proximal handpiece and optionally comprises a button or lever actuator, the latter being positioned at a rear portion of said proximal handpiece.

6. The circular surgical stapler according to claim 1, comprising approaching/spacing apart means, configured to determine an approaching or a spacing apart of said distal feeder and anvil for assuming said spaced apart configuration or a further spaced apart configuration, which approaching/spacing apart means is arranged at said proximal handpiece and optionally comprises a cursor-like actuator, which can be translated according to a longitudinal direction.

7. The circular surgical stapler according to claim 1, having a plurality of openings, obtained at an outer skirt of said feeder and each opening configured to allow the passage towards the outside of one or more traction wires applied to the prolapsed tissue.

8. The circular surgical stapler according to claim 1, wherein said proximal handpiece (110) comprises a substantially gun-shaped handgrip portion, which can be grasped by the operator with a palm of a hand.

9. The circular surgical stapler according to claim 1, comprising means for locking said anvil in a proximally retracted position.

10. The circular surgical stapler according to claim 1, wherein said second trigger is operatively connected to cutting means, so that actuation of the trigger also determines cutting of the prolapsed tissue.

11. A circular anal dilator, comprising:
an enlarged base configured to abut the anus; and
a hollow and oblong main body, having a substantially cylindrical geometry and extending along a longitudinal axis,
which main body is configured to be introduced in the rectum, in particular to perform a surgical procedure for treating the prolapse,
which main body has a lateral skirt defining an internal longitudinal cavity, wherein said lateral skirt is formed at least partially by a plurality of longitudinal sliding sectors which can be selectively retracted during a surgical procedure so as to allow insertion of tissues within said longitudinal cavity.
